(19) United States
(12) Patent Application Publication
(10) Pub. No.: US 2025/0072875 A1
(43) Pub. Date: Mar. 6, 2025

(54) SYSTEMS, METHODS, AND DEVICES FOR NEUROLOGICAL AND/OR MUSCULOSKELETAL PARAMETER CHARACTERIZATION

(71) Applicant: GS-HealthMatrix, LLC, Los Angeles, CA (US)

(72) Inventors: Joseph Schwab, Los Angeles, CA (US); Amirhossein Yazdkhasti, Los Angeles, CA (US); Hamid Ghaednia, Los Angeles, CA (US)

(73) Assignee: GS-HealthMatrix, LLC, Los Angeles, CA (US)

(21) Appl. No.: 18/819,415

(22) Filed: Aug. 29, 2024

Related U.S. Application Data
(60) Provisional application No. 63/579,605, filed on Aug. 30, 2023.
(60) Provisional application No. 63/579,616, filed on Aug. 30, 2023.
(60) Provisional application No. 63/579,627, filed on Aug. 30, 2023.
(60) Provisional application No. 63/579,633, filed on Aug. 30, 2023.
(60) Provisional application No. 63/579,640, filed on Aug. 30, 2023.
(60) Provisional application No. 63/579,647, filed on Aug. 30, 2023.
(60) Provisional application No. 63/579,663, filed on Aug. 30, 2023.

(51) Int. Cl. A61B 9/00; A61B 5/00; A61B 5/256; A61B 7/00; A61B 90/00

(52) U.S. Cl. CPC A61B 9/005; A61B 5/256; A61B 5/6831; A61B 5/742; A61B 7/006; A61B 2090/064

(57) ABSTRACT

Systems, methods, and devices include a neurological function assessment system involving a first assessment tool (e.g., hammer) with one or more first sensors. The system includes a second assessment tool being a wearable device operable to wrap around a body part of a subject. One or more second sensors are disposed on the wearable device operable to sense a target area at the body part. The system collects first sensor data, from the first assessment tool, corresponding to an assessment event at the first assessment tool; and collects second sensor data, from the second assessment tool, corresponding to the assessment event. Additionally, the system presents, at a display, an indication of an objective neurological assessment parameter value calculated based on the first sensor data and the second sensor data. An objective neurological assessment parameter value can be calculated based on the first sensor data and/or the second sensor data.

### CROSS-REFERENCE TO RELATED APPLICATIONS

This application claims priority to U.S. Provisional Application Ser. No. 63/579,605 filed Aug. 30, 2023 and titled “FRUSTRATED TOTAL INTERNAL REFLECTION (FTIR) SURFACE TOPOGRAPHY AND COMPOSITION ANALYSIS SYSTEMS, METHODS, AND DEVICES;” U.S. Provisional Application Ser. No. 63/579,616 filed Aug. 30, 2023 and titled “SYSTEMS, METHODS, AND DEVICES OF WEARABLE ELECTRO-ACOUSTIC MONITORING;” U.S. Provisional Application Ser. No. 63/579,627 filed Aug. 30, 2023 and titled “SYSTEMS, METHODS, AND DEVICES FOR ACOUSTICALLY ENHANCING IMPLANTS;” U.S. Provisional Application Ser. No. 63/579,633 filed Aug. 30, 2023 and titled SYSTEMS, METHODS, AND DEVICES WITH SENSORS HAVING MULTIPLE DETECTION SIGNAL TYPES;” U.S. Provisional Application Ser. No. 63/579,640 filed Aug. 30, 2023 and titled MULTI-DEVICE HEALTH PARAMETER MONITORING SYSTEMS, METHODS, AND DEVICES;” U.S. Provisional Application Ser. No. 63/579,647 filed Aug. 30, 2023 and titled FRUSTRATED TOTAL INTERNAL REFLECTION (FTIR)-BASED HEALTH PARAMETER DETECTION SYSTEMS, METHODS, AND DEVICES;” and U.S. Provisional Application Ser. No. 63/579,663 filed Aug. 30, 2023 and titled “SYSTEMS, METHODS, AND DEVICES FOR NEUROLOGICAL AND/OR MUSCOSKELETAL PARAMETER CHARACTERIZATION;” the entireties of which are herein incorporated by reference.

### BACKGROUND

Conventionally, a physician is trained to perform a reflex examination and understand the performance of the patient's musculoskeletal system based on multiple subjective observations. For instance, a tuning fork with resonance of 128 Hz and 256 Hz is placed on the boney prominence of a finger or toe. The physician must subjectively determine a placement and vibrating intensity for the test. The evaluation is completed by asking the patient about the sensation as vibration, another subjective determination. Because these analyses are subjective, they are often not based on accurate measurements or analysis of the patient's condition. Additionally, the patient's vibratory sense may be impaired or diminished due to aging or neurological or other types of diseases and conditions.

It is with these observations in mind, among others, that various aspects of the present disclosure were conceived and developed.

### SUMMARY

Systems, methods, and devices disclosed herein can address the aforementioned issues. For instance, a neurological function assessment system can include a first assessment tool being a reflex hammer including a handle, a hammer head, and/or one or more first sensors. The system can also include a second assessment tool being a wearable device. This wearable device can be a band operable to wrap around a body part of a subject. Also, the system can include one or more second sensors disposed on the band operable to sense a target area at the body part, and/or one or more non-transitory computer-readable media storing instructions which, when executed by one or more processors, cause the neurological function assessment system to perform one or more operation(s). The one or more operation(s) can include collecting first sensor data, from the first assessment tool, corresponding to an assessment event at the first assessment tool; collecting second sensor data, from the second assessment tool, corresponding to the assessment event; and/or presenting, at a display, an indication of an objective neurological assessment parameter value calculated based on the first sensor data and the second sensor data.

In some examples, the one or more first sensors can include one or more electrical sensors and one or more acoustic sensors. Additionally, the first sensor data cam represent a tissue activation occurring at the target area responsive to the assessment event. The tissue activation can include a muscle activation, a nerve activation, or a tendon activation. Also, the one or more second sensors can include at least one of an accelerometer, a gyroscope, a location sensor, a position sensor, a force sensor, a motion sensor, and/or any combination thereof. The second sensor data can represent a measured physical value of a swinging motion of the reflex hammer. Moreover, the assessment event can include a knee jerk reflex test performed with the reflex hammer; and/or the target area can include an upper leg area of the subject. Furthermore, the second assessment tool can include an intermediate contact portion operable to be placed over an area adjacent the target area. The intermediate contact portion can include one or more third sensors operable to measure an impact force of the reflex hammer. The first assessment tool can include one or more electrodes; and/or the first sensor data can include electrical activity data detected by the one or more electrodes.

In some scenarios, a neurological function assessment system can include a first assessment tool including one or more first sensors; and/or a second assessment tool being a wearable device. The wearable device can include a band operable to wrap around a body part of a subject, and/or one or more second sensors disposed on the band operable to sense a target area at the body part. The system can also include one or more non-transitory computer-readable media storing instructions which, when executed by one or more processors, cause the neurological function assessment system to perform one or more operation(s). These operation(s) can include collecting first sensor data, from the first assessment tool, corresponding to an assessment event including the first assessment tool contacting an area of the subject adjacent the target area; collecting second sensor data, from the second assessment tool, corresponding to the assessment event; and/or presenting, at a display, an indication of an objective neurological assessment parameter value calculated based on the first sensor data and the second sensor data.

In some instances, the first assessment tool can include a reflex hammer with a handle and a hammer head; and/or the one or more first sensors can be formed into the handle or the hammer head. The one or more first sensors can include at least one of an accelerometer, a gyroscope, a location sensor, a position sensor, a force sensor, or a motion sensor. Also, the first assessment tool can include a contact pad, positionable at the target area, and/or operable to measure an impact force. Furthermore, the one or more second sensors can include a plurality of electrical sensors forming an array on the band of the second assessment tool. For instance, the one or more second sensors can include a plurality of electro-acoustic sensors and a plurality of electro-acoustic actuators. Additionally, the instructions, when executed by the one or more processors, can cause the neurological function assessment system to collect third sensor data, from the second assessment tool, and responsive to an electric or acoustic stimulus caused by the second assessment tool. The instructions can also cause the objective neurological assessment parameter value to be calculated based at least partly on the third sensor data. The objective neurological assessment parameter value can be based on at least one of a reflex response value; a motor power response value, a sensation response value, and/or any combination thereof. Also, the first assessment tool can include a swab or a pinprick tool; and/or the assessment event can include a light touching with the first assessment tool at the area of the subject adjacent to the target area.

In some examples, a method of neurological function assessment includes collecting first sensor data, using a first assessment tool having one or more first sensors. The first sensor data can correspond to the first assessment tool contacting an area adjacent to a target area of a subject. The method can also include collecting second sensor data using a second assessment tool being a wearable device including one or more second sensors operable to sense the target area; and/or presenting, at a display, an indication of an objective neurological assessment parameter value calculated based on the first sensor data and the second sensor data.

In some scenarios, the method includes calculating, based on the first sensor data, an impact force associated with the first assessment tool contacting the area adjacent the target area. The objective neurological assessment parameter value can be calculated by using the impact force as a normalization factor. Moreover, the method can include calculating, based on the second sensor data, a tissue activation factor associated with a tissue of the target area. The objective neurological assessment parameter value can be calculated based on the tissue activation factor.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including one or more neurological and/or musculoskeletal function assessment devices.

FIGS. 2A and 2B illustrate an example system including one or more neurological and/or musculoskeletal function assessment devices using one or more neurological parameter values.

FIG. 3 illustrates an example system including one or more neurological and/or musculoskeletal function assessment devices using an assessment computation system to output an objective assessment value.

FIGS. 4A-4C illustrate example systems including one or more impact tools to perform a neurological and/or musculoskeletal function assessment.

FIG. 5 illustrates an example system including one or more wearable devices to perform a neurological and/or musculoskeletal function assessment.

FIG. 6 illustrates an example method of performing a neurological and/or musculoskeletal function assessment, which can be performed by any of the systems depicted in FIGS. 1-5.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein can be used to provide a quantitative functional and physical examination of a subject resulting in an objective neurological parameter characterization. The objective neurological parameter characterization can be based on one or more parameter category values such as a motor function parameter value, a sensation parameter value, and/or a reflex parameter value. The motor function parameter value can correspond to, for instance a determination of whether and to what extent the subject can move their arms, legs, or, body against gravity, and/or whether and to what extent the subject can move their joints against resistant applied by a test facilitator (e.g., a physician). The sensation parameter value can correspond to a response to a tactile stimulus, such as a pinprick or cotton swab used by the test facilitator (e.g., the physicians or experts) to examine the subject's neurological system performance or muscle/nerve activations with respect to tactile sensation. One example includes examining a progress assessment of the neurological system following surgery using a series of pinpricks over time and quantifying the measured electrical/acoustic responses. The reflex parameter value can be based on an impact response, such as a response to an impact hammer.

In some scenarios an expert physician is trained to perform an examination and understand the performance of the patient's musculoskeletal system using human observations. For instance, a test facilitator may use a tuning fork with resonances of 128 Hz and/or 256 Hz placed on a boney prominence of a finger of toe. The evaluation can be completed by asking the subject about their experienced sensation as the tuning fork vibrates because their vibratory sense may be impaired or diminished due to aging or neurological or other types of diseases and conditions. However, these analysis are subjective, use inconsistent inputs (e.g., an amplitude of the tuning fork vibrations), and are not based on accurate measurements and analysis of the patient condition.

The technology disclosed herein supplements and/or replaces the subjective aspects of the assessment with a quantitative, objective output. Moreover, the systems disclosed herein can generate precise stimulations, such as vibrations with different particular frequencies and intensities, to investigate vibratory sensation with higher accuracy, and by carefully measuring applied force. Additional advantages and benefits of the presently disclosed technology will become apparent from the detailed description below.

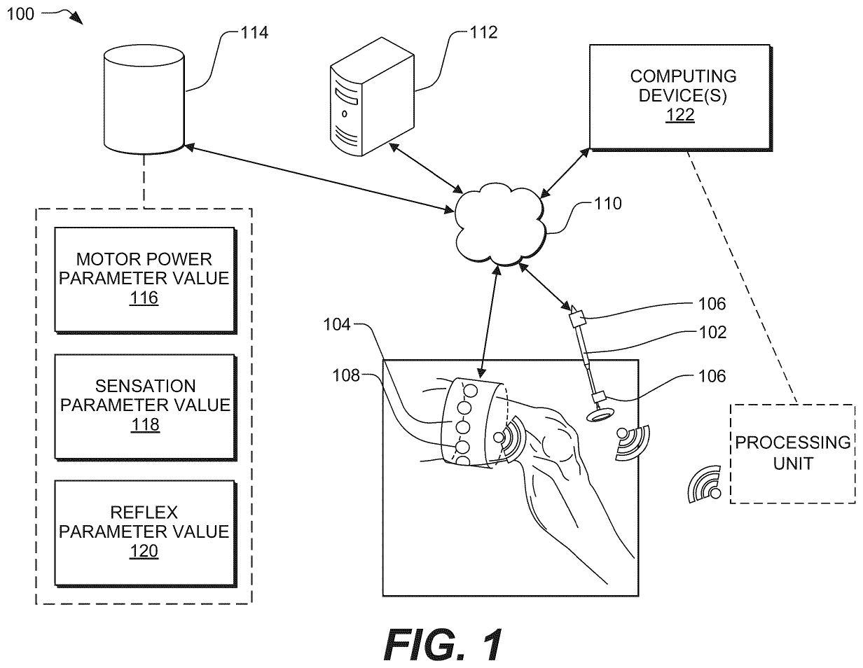

FIG. 1 depicts an example neurological function assessment system 100 for performing an objective neurological and/or musculoskeletal function assessment of a subject. The neurological function assessment system 100 can include a first assessment tool 102 operable to provide an assessment stimulus and a second assessment tool 104 for detecting a signal responsive to the stimulus (e.g., a “stimulus response”). The first assessment tool 102 can also at least partly detect the stimulus response, and/or the second assessment tool 104 can at least partly provide some or all of the assessment stimulus in various combinations, as discussed in greater detail below.

In some examples, the first assessment tool 102 can include an excitation or stimulation device with one or more first sensors 106. The excitation or stimulation device can include one or more of a variety of devices for generating stimulus to a human or animal body and causing the stimulus response from the body (e.g., the musculoskeletal system). For instance, the excitation or stimulation device can include an impact tool, such as a hammer and/or a punch/contact pad. The excitation or stimulation device could also include a touching tool (e.g., a swab, a pin prick, or an electrode) or a resistance tool (e.g., an exercise machine or a flexible band) or any combination thereof. Moreover, the excitation/stimulation device can include a wearable cuff or sleeve with a variety of sensors and/or actuators, such as the second assessment tool 104 discussed in greater detail below.

These devices can provide excitation/stimulation to stimulate a particular tendon, muscle, and/or or nerve by applying a controlled and quantitatively measurable force, prick, touch, vibration, or so forth, and by measuring the applied stimulus (e.g., force, velocity, position, acceleration, tilt, frequency, temperature, etc.) The stimulus characteristics (and/or the forces applied with these devices can be measured using a built-in sensing system of the first assessment tool 102 itself. This sensing system can provide information about the test facilitator's performance in applying the assessment stimulus to the subject. For instance, the sensing system can provide information about the stimulus properties including one or more of timing, applied force, contact location, velocity, acceleration, position relative to another device, tilt, angle of contact, transmitted energy, and so forth. The first assessment tool 102 can also have electrical sensing capabilities to find the proximity to nerves and measure contact time. Any of these components can be integrated into the moving portion(s) of the first assessment tool 102 (e.g., the hammer head, the handle, etc.) and/or into a separate portion, such as an intermediate contact portion (e.g., punch or a contact pad) laid over the target area of the subject to be struck by the impact tool. If the neurological function assessment system 100 determines any of these measurements fails to satisfy a predetermined threshold value, the first assessment tool 102 can indicate via an audio/visual notification that the assessment stimulus failed to satisfy the predetermined threshold value and/or the assessment stimulus should be repeated.

In some instances, the second assessment tool 104 includes one or more second sensors 108, which can be any combination of electrical sensors and/or acoustic sensors (e.g., electroacoustic sensors). Moreover, the first assessment tool 102 can include any combination of electrical actuators and/or acoustic actuators (e.g., electroacoustic actuators) for generating and sending electrical and/or acoustic signals into the target region around which the second assessment tool 104 is placed. The electrical and/or acoustic sensing system can evaluate responses of nerves and/or motor systems to the assessment stimulation. Accordingly, the second assessment tool 104 can quantitatively measure performance of the neurological and musculoskeletal system instead of or supplemental to a subjective grading by a professional operator/physician.

In some instances, the first assessment tool 102 and/or the second assessment tool 104 can include one or more network interfaces for establishing a wired or wireless connection with one or more networks 110. In other words, the neurological function assessment system 100 can receive and/or transmit data to and from the various components of the neurological function assessment system 100 using the network(s) 110 and the corresponding network connections. The network 110 can include any type of network, such as the Internet, an intranet, a Virtual Private Network (VPN), a Voice over Internet Protocol (VoIP) network, a wireless network (e.g., Wi-Fi, Bluetooth), a local exchange carrier (LEC) network, a cellular network (e.g., 4G, 5G, LTE, etc.), satellite, wired networks (e.g., ethernet, fiber, a public switched telephone network (PSTN)) combinations thereof, etc. The network 110 can include communications network(s) with numerous components such as, but not limited to gateways routers, servers, and registrars, which enable communication across the network 110. In some implementations, the network(s) 110 includes multiple ingress/egress routers, which may have one or more ports, in communication with the network 110.

The neurological function assessment system 100 can also include at least one server 112 and/or one or more other computing device 122 hosting a website or application that the first assessment tool 102 and/or the second assessment tool 104 may visit to access the components of the neurological function assessment system 100. The server 112 and/or the one or more other computing device 122 can access (e.g., read and/or write) one or more database(s) 114. The database(s) 114 can store one or more of a motor power parameter value 116, a sensation parameter value 118, and/or a reflex parameter value 120, which can be numerical values outputted and/or retrieved by the computing device(s) 122 of the neurological function assessment system 100. The website or application can receive the inputs from the first assessment tool 102, the second assessment tool 104, and/or one or more other computing device 122, and can analyze the inputs to generate outputs for the neurological function assessment system 100 (e.g., which can be stored at the database(s) 114 and/or displayed on any of the devices).

The server 112 may be a single server, a plurality of servers with each such server being a physical server or a virtual machine, or a collection of both physical servers and virtual machines. In another implementation, a cloud hosts one or more components of the neurological function assessment system 100. The server 112 may represent an instance among large instances of application servers in a cloud computing environment, a data center, or other computing environment. The server 112 can access the data stored at the one or more database(s) 114. The first assessment tool 102, the second assessment tool 104, the one or more other computing device 122 and/or other resources connected to the network 110 may access the database(s) 114 and one or more other servers to access one or more websites, applications, web services interfaces, storage devices, computing devices, or the like.

Furthermore, the one or more other computing device 122 can include a computer, a personal computer, a desktop computer, a laptop computer, a terminal, a workstation, a cellular or mobile phone, a mobile device, a smart mobile device, a tablet, a wearable device (e.g., a smart watch, smart glasses, a smart epidermal device, etc.), a multimedia console, a television, an Internet-of-Things (IoT) device, a smart home device, a virtual reality (VR) or augmented reality (AR) device, another server device, a vehicle, and/or combinations thereof.

The one or more database(s) 114 can be implemented in one or more memory device(s), such as any non-volatile data storage device capable of storing data generated or employed by the neurological function assessment system 100. Theses memory storing devices can store computer executable instructions for performing (e.g., when executed by one or more processors) any of the computer processes discussed herein, which may include instructions of both application programs and an operating system (OS) that manages the various components of the devices. The memory device(s) may include, without limitation, magnetic disk drives, optical disk drives, solid state drives (SSDs), flash drives, and the like. The memory device(s) may include removable data storage media, non-removable data storage media, and/or external storage devices made available via a wired or wireless network with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) may include volatile memory (e.g., dynamic random-access memory (DRAM), static random-access memory (SRAM), etc.) and/or non-volatile memory (e.g., read-only memory (ROM), flash memory, etc.).

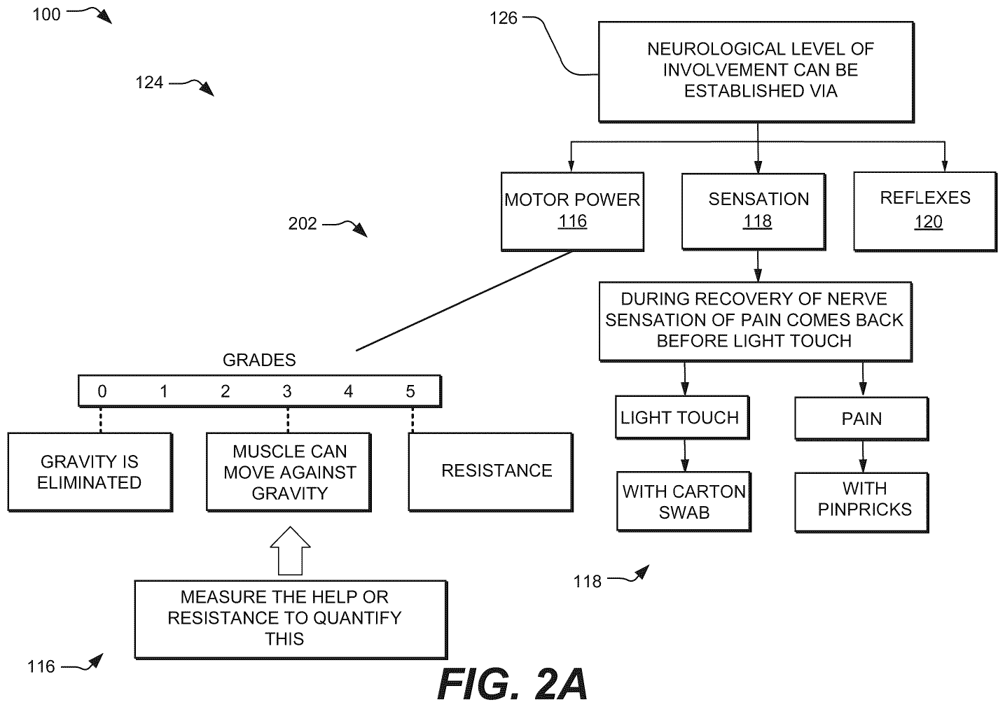

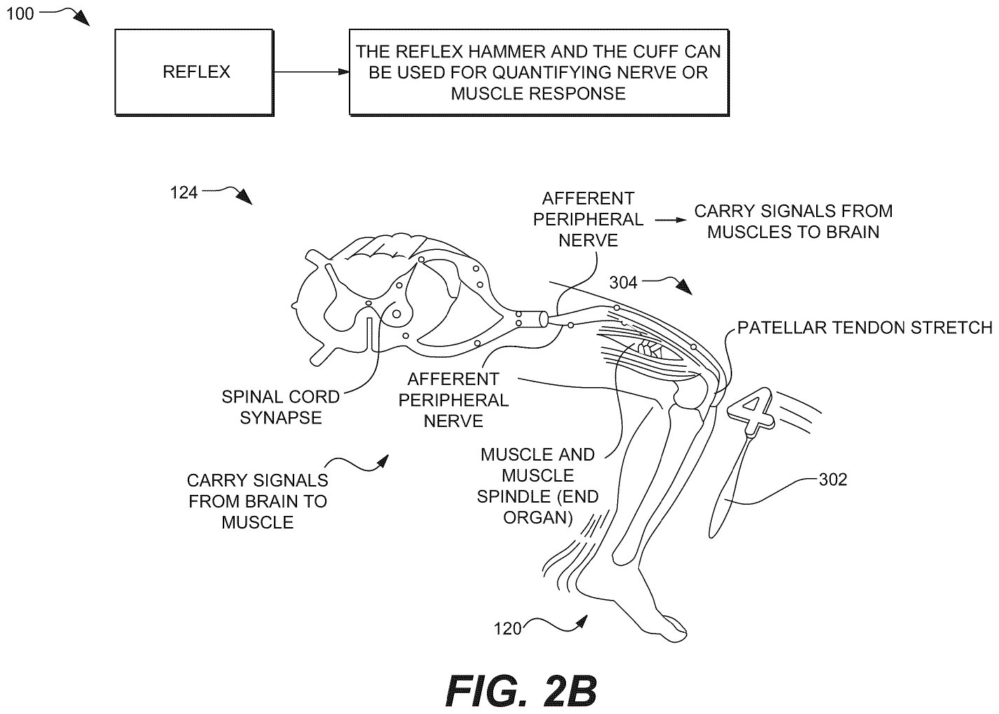

FIGS. 2A and 2B illustrate an example diagram of an objective neurological parameter characterization 202 which can be generated by the neurological function assessment system 100 disclosed herein. For instance, the objective neurological parameter characterization 202 can be based on data collected for one or more parameter category values, such as the motor function parameter value 116, the sensation parameter value 118, and/or the reflex parameter value 120. These parameter values can include one or more numerical ratings or scale values determined based on the data provided by the one or more first sensors 106 of the first assessment tool 102 and/or the one or more second sensors 108 of the second assessment tool 104. For instances, the motor function parameter value 116 can be on a low-to-high scale with no resistance or gravity at one end of zero and maximum resistance at the other (e.g., a “5,” “10,” or “100”). Moreover, the sensation parameter value 118 can include a numerical value, based on the data corresponding to an amount of touch or pain response collected by the first assessment tool 102 and/or the second assessment tool 104. Finally, the reflex parameter value 120 can include a numerical value corresponding to the data collected by the first assessment tool 102 and/or the second assessment tool 104, such as an electrical signal and/or an acoustic signal detected from the afferent peripheral nerve activation, the efferent peripheral nerve activation, a muscle movement, a tendon movement/stretch (e.g., at the patella), combinations thereof, and the like.

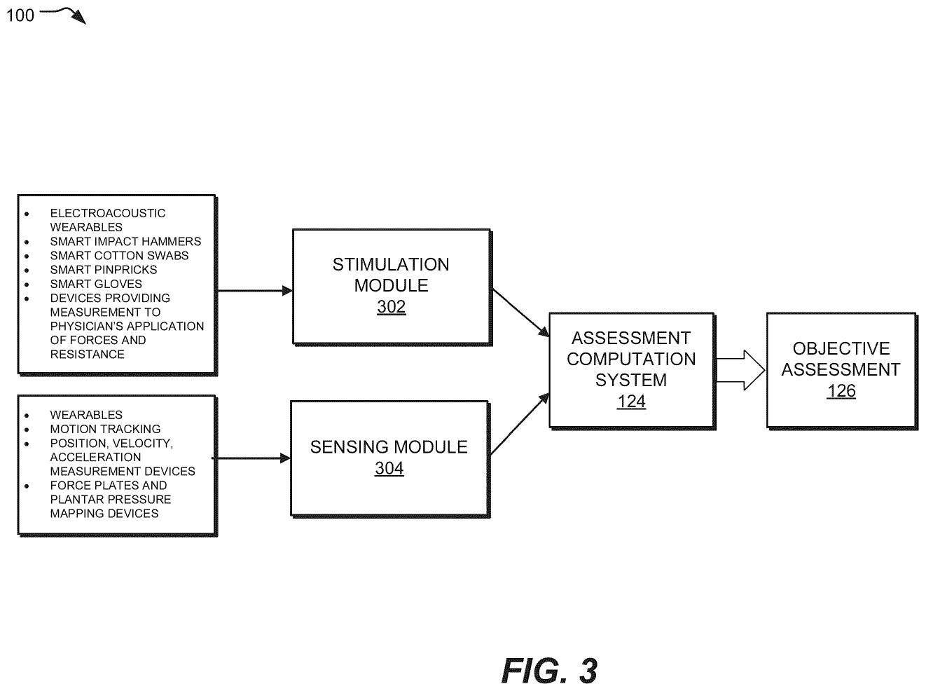

FIG. 3 depicts an example diagram of the neurological function assessment system 100 including the first assessment tool 102 and the second assessment tool 104, which can provide their collected data to an assessment computation system 124 for generating the objective assessment 126. The neurological function assessment system 100 depicted in FIG. 3 can form at least a portion of neurological function assessment system 100 depicted in FIG. 1.

In some example, the first assessment tool 102 can form part of a stimulation module 302 which can include a Queen square hammer (e.g., Vernon or Wintle), a Taylor tomahawk hammer, a sensing punch, a vibratory sense testing device, an electroacoustic wearable, a cotton swab, a smart glove, a pinprick, combinations thereof, or other devices providing measurement for the physician's application of forces and resistance. Furthermore, the second assessment tool 104 can form part of a sensing module 304 which can include various sensing devices such as a single electrical of acoustic sensor, an array of acoustic and/or electrical sensors, a wearable device (e.g., cuff, sleeve, strap, band, cap, glove, etc.), a motion measurement device, a position measurement device, a velocity measurement device, an acceleration measurement device, a force plate, a plantar pressure mapping device, and/or combinations thereof.

In some examples, the assessment computation system 124 can include the computer-executable instructions operating on the one or more other computing device 122. For instance, the assessment computation system 124 can execute one or more machine-learning programs to receive the data from the first assessment tool 102 and the second assessment tool 104 and can generate the objective assessment 126. The assessment computation system 124 can undergo one or more training operations in which historical sensor data collected by subjects undergoing the assessment stimulation is provided and/or labeled. Furthermore, the assessment computation system 124 can combine different types of data signals (e.g., acoustic signals, electrical signals), and/or can use motion data from the first assessment tool 102 to normalize the stimulus response data.

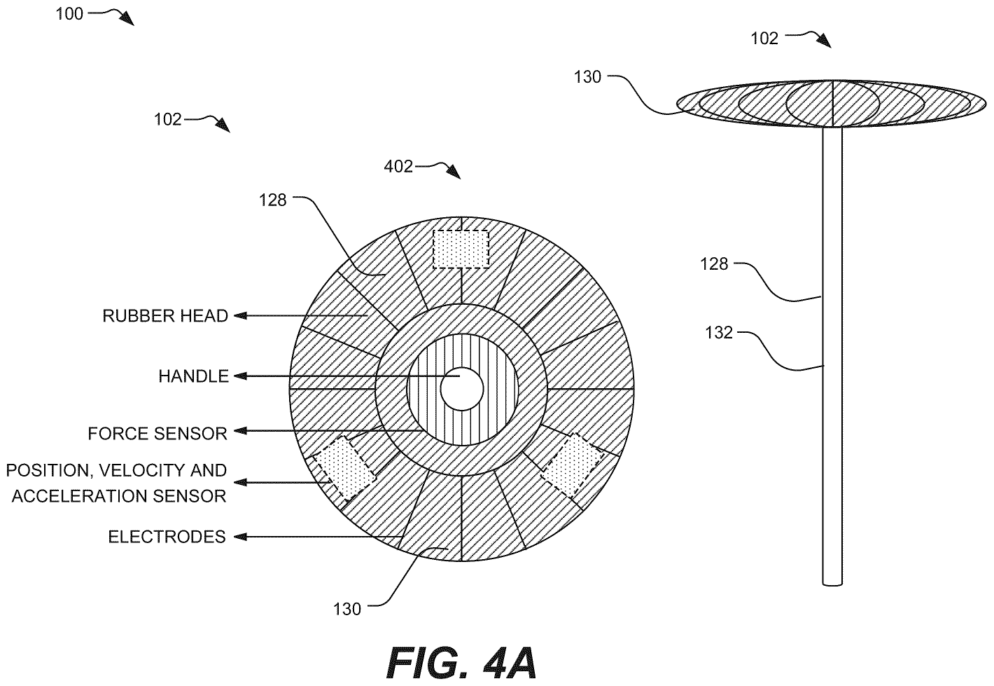

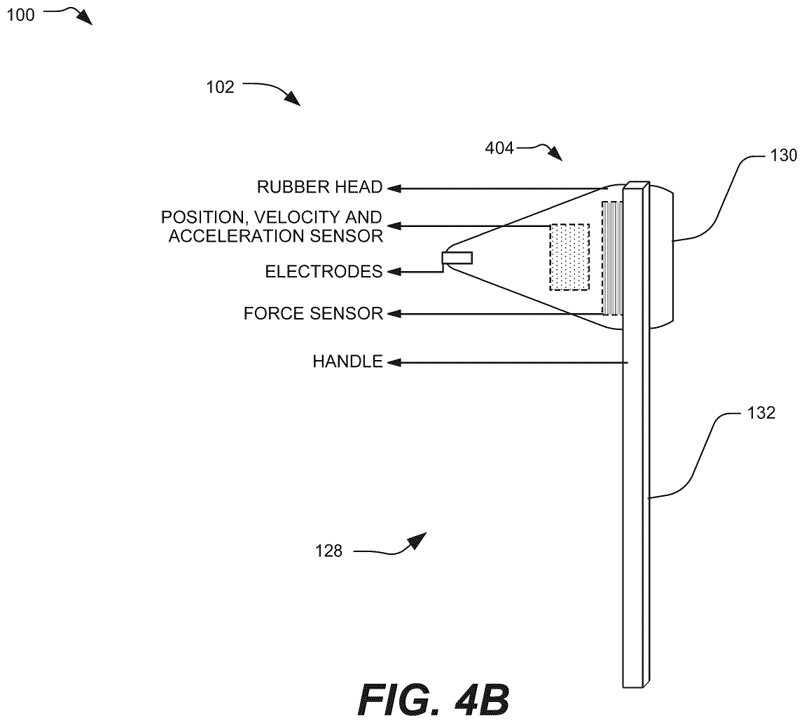

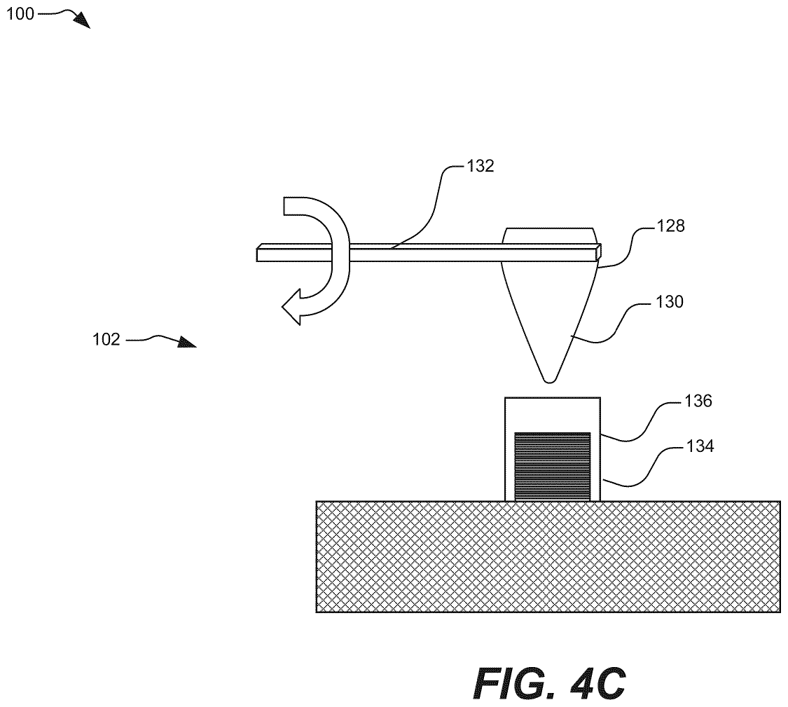

FIGS. 4A-4C depict examples systems 100 including the first assessment tool 102, which can be an impact tool such as a reflex hammer 128. The reflex hammer 128 can include a hammer head portion 130 (e.g., formed of rubber) defining a first end and/or a handle portion 132 extending from the hammer head portion 130 to define a second end. The hammer head portion 130 and/or the handle portion 132 can include the one or more first sensors 106, such as a force sensor, a position sensor, a velocity sensor, an acceleration sensor, and/or one or more electrodes. The reflex hammer 128 can integrate three-axis force sensors, position, velocity, and/or acceleration sensors. The electrical electrodes could be used for one or more of locating nerves, measurement of the impact duration, and/or measurements of the stimulus response. As shown in FIG. 4A, the hammer head portion 130 can be round and/or disc shaped 402. Furthermore, as shown in FIG. 4B the hammer head portion 130 can have a triangular side profile 404, for instance, with the electrode disposed at a tip end of the triangle.

FIG. 4C depicts an example first assessment tool 102 in which one or more first sensor 106, such as the force sensor, is not included in the hammer 128 itself and is included in an intermediate contact portion 134, such as a punch 136 placed on the target contact area, and then the reflex hammer 128 can hit the punch 136 instead of directly hitting the body. The one or more first sensors 106 can be disposed only in the punch 136, only in the reflex hammer 128, or in both the punch 136 (e.g., and/or the contact pad) and the reflex hammer 128, such that data can be collected and compared from multiple data sets for improved accuracy and output optimization.

Furthermore, as noted above, the first assessment tool 102 can include a sensation tool with the one or more first sensors 106 such as a cotton swab integrating multiple different sensors (e.g., force, position, velocity, acceleration, pressure, heat, surface contact, etc.) that provide quantitative measurements about the assessment stimulus provided by the test facilitator to ensure the objective assessment 126 of the patient's reaction.

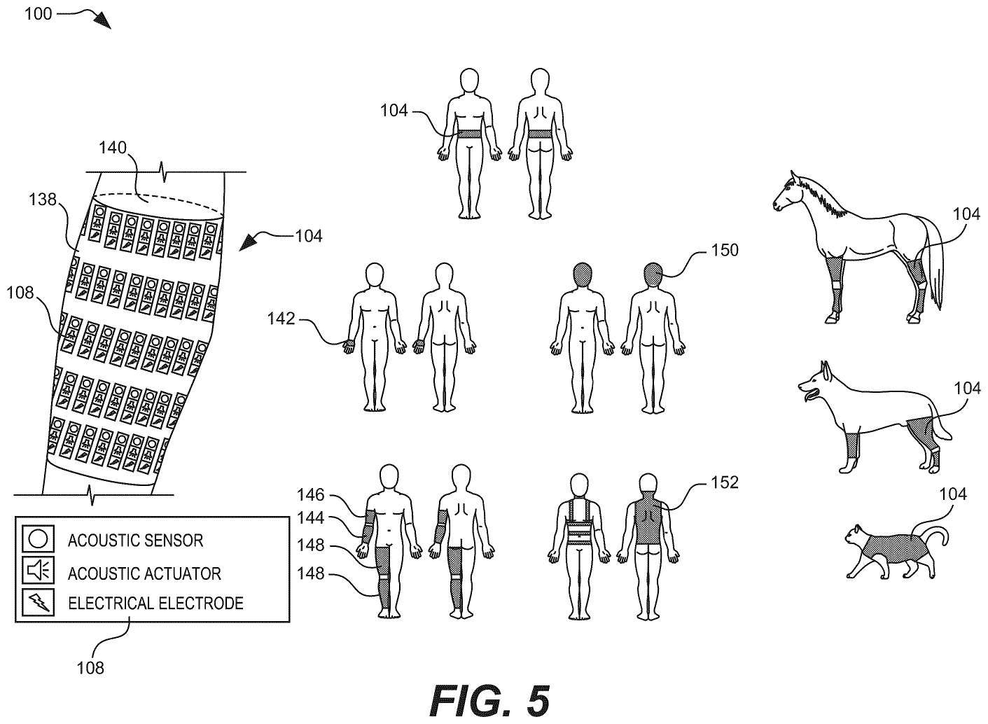

FIG. 5 depicts an example second assessment tool 104 which can include a wearable device 138 with a plurality of sensors. The system(s) 100 depicted in FIG. 4 can form at least a portion of the system 100 depicted in FIG. 1.

In some examples, the second assessment tool 104 disclosed herein can be capable of independent acoustic and electrical sensing and actuating simultaneously. At the same time, the second assessment tool 104 can be used to stimulate the body in one of the electrical domain or the acoustic domain, to supplement and/or substitute the assessment stimulus provided by the first assessment tool 102, and to monitor the reaction of the body using the other domain. The second assessment tool 104 can include an inner surface 140 that forms a measurement surface for detecting the stimulus response.

Accordingly, the second assessment tool 104 disclosed herein can measure a variety of stimulus responses at different target regions of the user. The second assessment tool 104 can be used to measure the muscle activity response, the nerve activity response, and/or the tendon response to the assessment stimulus. Moreover, the second assessment tool 104 can measure a muscle activity, a muscle health, and/or a muscle healing process over a period of time, and this data can be used to determine the objective assessment 126 (e.g., as normalization data). Additionally, the neurological function assessment system 100 disclosed herein can be used to monitor bone density, water content, and/or a bone healing process with the second assessment tool 104. Also, the neurological function assessment system 100 can monitor and/or detect an implant and/or how the implant impacts the response to the assessment stimulus (e.g., compared to datasets of subject omitting the implant).

In some examples, the second assessment tool 104 can be formed of fabric, plastic, or other flexible or partially flexible materials and/or various other types of material to form different shapes, sizes, and form-factors. The second assessment tool 104 can also include the one or more second sensors 108 and/or a plurality of actuators. The plurality of sensors/actuators can include any combination of acoustic actuators, acoustic sensors, electrical actuators, and/or electrical sensors. The second assessment tool 104 can include one or more integrated sensors/actuators, the integrated sensor/actuator being a combination of the acoustic sensor, the acoustic actuator, and/or the electrical electrode. The second assessment tool 104 can include an array of the integrated sensors. The wearable(s) device 104 can also include a power source, such as a battery and/or an AC power adapter disposed on the second assessment tool 104 (or separate from the second assessment tool 104). The second assessment tool 104 can also include a controller, such as a processor or microcontroller, for implementing a sensing control system, an actuation control system, and/or a sensing analytics engine. The controller and/or any components of the controller can be integral with the second assessment tool 104 (e.g., disposed on the second assessment tool 104), or the controller and/or any components of the controller can be remote or separate from the second assessment tool 104, such as at the first assessment tool 102, the server 112 and/or the one or more other computing device 122. The sensing analytics engine can perform one or more sensor data analyses and/or cross-domain analyses using the sensor data collected from the second assessment tool 104 as well as from the first assessment tool 102.

In some instances, the second assessment tool 104 can be an acoustic-electric assessment tool which generates acoustic and electrical signals to supplement, modify, and/or substitute at least a portion of the assessment stimulus. These signals can have different waveforms (e.g., pure tone, gaussian waves, or so forth), different frequencies (e.g., 1 Hz to 10 MHz), and/or different intensities from the sensors/actuators. The generated signals can interact with soft and hard tissues. These interactions may lead to transformation of tissues or transformation of wave. The transformed wave can be measured using the same or a different array of electrical and acoustic sensors and can be provided to the assessment computation system 124 for generating the objective assessment 126 (e.g., as an initialization/calibration process and/or a data collection process). Furthermore, the plurality of sensors/actuators can include one or more piezoelectric transducer used for momentous sensing and actuating, and/or one or more micro-electromechanical system (MEMS) microphones and/or speakers, which can be used for denser arrays. The second assessment tool 104 can also include a small-scale FTIR-based sensor and/or any of the components of the first assessment tool 102 formed as a micrometer, millimeter, or centimeter scale sensor assembly on the base material of the second assessment tool 104. The sensing analytics engine can include one or more machine learning (ML) models for extracting information from the acoustic, electrical, thermal, and/or optical signals measured at the second assessment tool 104 (e.g., using various architectures to perform a time series analysis).

In some examples, the second assessment tool 104 can form one or more cuffs or sleeves configured to at least partially cover and/or wrap around particular target areas or regions of the body. For example, the second assessment tool 104 can include one or more of a hand sleeve 142, a wrist sleeve 144, an arm sleeve 146 (e.g., an upper arm sleeve and/or a lower arm sleeve), a leg sleeve 148 (e.g., an upper leg sleeve and/or a lower leg sleeve), a torso band, a head cap 150, a back strap, a chest strap, a back harness 152, and/or any combinations thereof. Furthermore, in scenarios where the neurological function assessment system 100 is used for animal husbandry, the second assessment tool 104 can form an animal leg sleeve and/or a body harness operable for use with a horse, a dog, a cat, or so forth.

In some examples, an acoustic signal and/or an electrical signal from the second assessment tool 104 can be detected at the first assessment tool 102 (e.g., via the electrode on the first assessment tool 102). Moreover, a signal actuation (e. a trigger signal) generated at the first assessment tool 102 can be detected at the second assessment tool 104. In some examples, both the first assessment tool 102 and the second assessment tool 104 can be wearable devices, such as electroacoustic wearable devices. For instance, the neurological function assessment system 100 could use two or more electroacoustic wearables with one being used for generating the assessment stimulation and the other one having the sensors configured to sense the reflex response, the sensation response, and/or the motor function response to the assessment stimulation. In another example, the neurological function assessment system 100 could use one or more electroacoustic wearables or other stimulation devices to operate as the first assessment tool 102, and one or more force plates and/or pressure mapping devices to measure motor and/or neurological system function response as the second assessment tool 104 as the subject is standing on, holding, or otherwise contacting the force place. An example could be a spherical pressure mapping device for measuring pressure on the subject's fingers while a wearable devices around the wrist or arm or spine provides stimulation to specific nerves. The stimulation could, additionally or alternatively, also be provided by the vibration forks, reflex hammers, pinpricks, cotton swabs and/or other means of stimulation discussed herein. Another example of the neurological function assessment system 100 can include a combination of motion tracking systems as the sensing module and various different stimulation devices as the stimulation module. For example, the reflex hammer 128 and the wearable devices 138 could be used for stimulation of nerves, and/or motion tracking cameras with or without markers or accelerometers could be used for sensing the stimulus response.

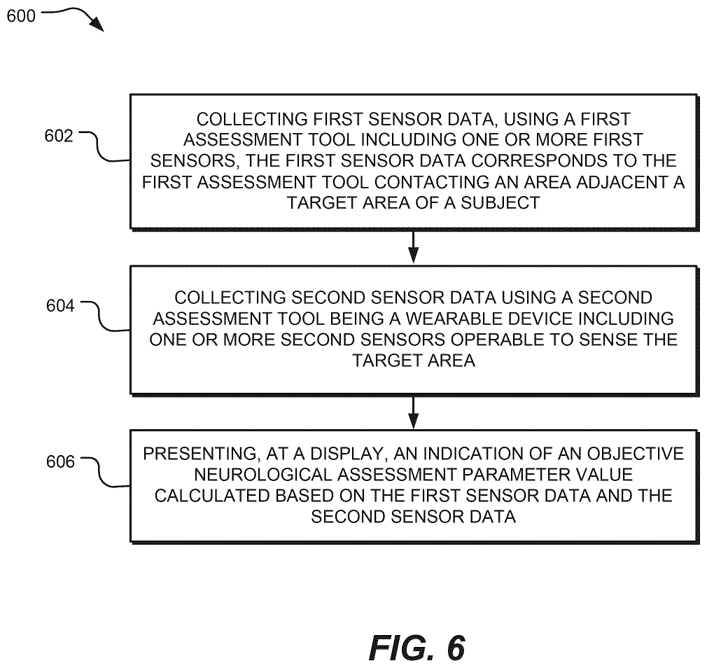

FIG. 6 depicts an example method 600 for performing a neurological function assessment using one or more assessment tools, which can be performed by any of the system(s) 100 depicted in FIGS. 1-5.

In some examples, at operation 602, the method 600 can collect first sensor data, using a first assessment tool including one or more first sensors, the first sensor data corresponds to the first assessment tool contacting an area adjacent a target area of a subject. At operation 604, the method 600 can collect second sensor data using a second assessment tool being a wearable device including one or more second sensors operable to sense the target area. At operation 606, the method 600 can present, at a display, an indication of an objective neurological assessment parameter value calculated based on the first sensor data and the second sensor data.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A neurological function assessment system comprising:
a first assessment tool being a reflex hammer including a handle, a hammer head, and one or more first sensors;
a second assessment tool being a wearable device including:
a band operable to wrap around a body part of a subject, and
one or more second sensors disposed on the band operable to sense a target area at the body part; and
one or more non-transitory computer-readable media storing instructions which, when executed by one or more processors, cause the neurological function assessment system to:
collect first sensor data, from the first assessment tool, corresponding to an assessment event at the first assessment tool,
collect second sensor data, from the second assessment tool, corresponding to the assessment event, and
present, at a display, an indication of an objective neurological assessment parameter value calculated based on the first sensor data and the second sensor data.

2. The system of claim 1,
wherein,
the one or more first sensors include one or more electrical sensors and one or more acoustic sensors.

3. The system of claim 2,
wherein,
the first sensor data represents a tissue activation occurring at the target area responsive to the assessment event.

4. The system of claim 3,
wherein,
the tissue activation includes a muscle activation, a nerve activation, or a tendon activation.

5. The system of claim 1,
wherein,
the one or more second sensors include at least one of an accelerometer, a gyroscope, a location sensor, a position sensor, a force sensor, or a motion sensor.

6. The system of claim 5,
wherein,
the second sensor data represents a measured physical value of a swinging motion of the reflex hammer.

7. The system of claim 1,
wherein,
the assessment event includes a knee jerk reflex test performed with the reflex hammer; and
the target area includes an upper leg area of the subject.

8. The system of claim 7,
wherein,
the second assessment tool includes an intermediate contact portion operable to be placed over an area adjacent the target area, the intermediate contact portion including one or more third sensors operable to measure an impact force of the reflex hammer.

9. The system of claim 1,
wherein,
the first assessment tool includes one or more electrodes; and
the first sensor data includes electrical activity data detected by the one or more electrodes.

10. A neurological function assessment system comprising:
a first assessment tool including one or more first sensors;
a second assessment tool being a wearable device including:
a band operable to wrap around a body part of a subject, and
one or more second sensors disposed on the band operable to sense a target area at the body part; and
one or more non-transitory computer-readable media storing instructions which, when executed by one or more processors, cause the neurological function assessment system to:
collect first sensor data, from the first assessment tool, corresponding to an assessment event including the first assessment tool contacting an area of the subject adjacent the target area,
collect second sensor data, from the second assessment tool, corresponding to the assessment event, and
present, at a display, an indication of an objective neurological assessment parameter value calculated based on the first sensor data and the second sensor data.

11. The system of claim 10,
wherein,
the first assessment tool includes a reflex hammer with a handle and a hammer head; and
the one or more first sensors are formed into the handle or the hammer head, and the one or more first sensors include at least one of an accelerometer, a gyroscope, a location sensor, a position sensor, a force sensor, or a motion sensor.

12. The system of claim 10,
wherein,
the first assessment tool includes a contact pad, positionable at the target area, and operable to measure an impact force.

13. The system of claim 10,
wherein,
the one or more second sensors includes a plurality of electrical sensors forming an array on the band of the second assessment tool.

14. The system of claim 10,
wherein,
the one or more second sensors includes a plurality of electro-acoustic sensors and a plurality of electro-acoustic actuators.

15. The system of claim 10,
wherein,
the instructions, when executed by the one or more processors, cause the neurological function assessment system to collect third sensor data, from the second assessment tool, and responsive to an electric or acoustic stimulus caused by the second assessment tool; and
the objective neurological assessment parameter value is calculated based at least partly on the third sensor data.

16. The system of claim 10,
wherein,
the objective neurological assessment parameter value is based on at least one of a reflex response value; a motor power response value, or a sensation response value.

17. The system of claim 10,
wherein,
the first assessment tool includes a swab or a pinprick tool; and
the assessment event includes a light touching with the first assessment tool at the area of the subject adjacent to the target area.

18. A method of neurological function assessment, the method comprising:
collecting first sensor data, using a first assessment tool including one or more first sensors, the first sensor data corresponds to the first assessment tool contacting an area adjacent a target area of a subject;
collecting second sensor data using a second assessment tool being a wearable device including one or more second sensors operable to sense the target area; and
present, at a display, an indication of an objective neurological assessment parameter value calculated based on the first sensor data and the second sensor data.

19. The method of claim 18, further comprising:
calculating, based on the first sensor data, an impact force associated with the first assessment tool contacting the area adjacent to the target area, the objective neurological assessment parameter value is calculated by using the impact force as a normalization factor.

20. The method of claim 18, further comprising:
calculating, based on the second sensor data, a tissue activation factor associated with a tissue of the target area, the objective neurological assessment parameter value is calculated based on the tissue activation factor.
